(19) United States
(12) Patent Application Publication
(10) Pub. No.: US 2001/0046665 A1
(43) Pub. Date: Nov. 29, 2001

(54) Method of identifying the function of a test agent

(76) Inventors: Thomas J. Powell, Madison, CT (US); Stacey A. Minskoff, Stamford, CT (US); Kerry E. Quinn, Hamden, CT (US); Tennore M. Ramesh, New Milford, CT (US)

Correspondence Address: Ivor R. Elrifi, Mintz, Levin, Cohn, Ferris,, Glovsky and Popeo, P.C., One Financial Center, Boston, MA, 02111, US

(21) Appl. No.: 09/766,863

(22) Filed: Jan. 19, 2001

Related U.S. Application Data
(60) Provisional application No. 60/177,416, filed on Jan. 21, 2000.

(51) Int. Cl. C12Q 1/68; C12Q 1/00

(52) U.S. Cl. 435/4; 435/6

(57) ABSTRACT

Disclosed is a method of identifying the function of a test compound by contacting a plurality of cells with the test compound. The plurality includes at least a first cell and a second cell of a different type than the first cell type. Expression of one or more genes in cells of the plurality is measured. An alteration in the expression of the genes relative to the expression of said one or more genes in a reference cell reveals the function of said test compound

### RELATED APPLICATIONS

This application claims priority to U.S. Ser. No. 60/177,416, filed Jan. 21, 2000. The contents of this application are incorporated herein by reference in their entirety.

### FIELD OF THE INVENTION

The invention relates to biochemistry, molecular biology, and cell biology.

### BACKGROUND OF THE INVENTION

The accumulation of raw nucleic acid sequence information for various organisms, coupled with the development of methods for identifying open reading frames encoding candidate proteins, is creating a need for methods that determine the function of previously unknown proteins. To date, functions of unknown proteins can be inferred by identifying genes whose expression changes (by increasing or decreasing) in the presence of the agent protein. However, such gene expression assays can be costly and labor-intensive. An effective and economical method for screening novel proteins for functions of interest is needed in the art.

### SUMMARY OF THE INVENTION

The invention is based in part on the discovery of a system and method for rapidly and economically identifying the function of a test agent, such as a polypeptide, by examining changes in expression of genes in a plurality of cells contacted with the test agent.

In one aspect, the invention includes a method of identifying the function of a test compound by contacting a plurality of cells with a test compound. The plurality includes at least a first cell and a second cell of a different type than the first cell type. Expression of one or more genes in cells or the plurality is measured. An alteration in the expression of the genes relative to the expression genes in a reference cell reveals the function of the test compound. For example, if the test compound is a polypeptide and induces a gene expression pattern characteristic of a cytokine, the test compound is considered a candidate new cytokine.

Preferably, the plurality includes three, four, five, six, or ten or more distinct cell types. Preferably, the expression of multiple genes, e.g., at least two, three, four, five, seven, and even ten genes is measured in one or more of the distinct cells in the array.

For example, the method can include measuring the expression of at least two genes (and more preferably at least five genes) in the first cell, and, optionally measuring the expression of at least two genes (and more preferably at least five genes) in the second cell. In a preferred embodiment, expression of one or more genes is also measured in a third cell, wherein the third cell is a different cell type from the first cell and the second cell. In a more preferred embodiment, expression of one or more genes is also measured in a fourth cell, wherein the fourth cell is a different cell type from the first cell, the second cell type, and the third cell type.

Expression of a gene or genes in a cell exposed to a test agent can be compared to expression of the gene in a reference cell (e.g., otherwise identical cells not exposed to the test agent). The reference cell may be processed in parallel to cells in the plurality; alternatively, expression information for the reference cell can be stored in a database.

The plurality of cells is preferably provided in a container in which different cell types in the plurality are spatially segregated. A preferred container is one in which the test agent can be added to the cells, after which the cells are lysed for isolating RNA. The container may in addition include control cells, e.g., cells not exposed to a test agent.

Examples of suitable test compounds include small molecules (typically molecules with molecular weights less than 1000 kDa) or larger macromolecules such as polynucleotides (including ribozymes) and polypeptides. Suitable polypeptides can also include antibodies. In some embodiments, two or more test compounds are added to the plurality of cells.

While any kind of cell can be used in the method, preferred cells are mammalian (e.g., human) cells. Cells can be from established cell lines, or can be primary cells. Cell lines used in the method are preferably derived from multiple tissue types. Cell lines may be growth factor dependent or growth factor independent. Test compounds may be added in the presence or absence of serum. Cell lines may be derived from tissues of different species, but are preferably mammalian cells. Most preferably, the cells are derived from human cells. The cell can be derived from a human tissue, i.e., a primary cell, or can be from an established (e.g., immortalized) cell line.

Examples of cells suitable for use in the invention include MG-63 cells, U87-MG cells, TF-1 cells, HepG2 cells, THP-1 cells, HUVEC cells, CCD-1070SK cells, and Jurkat E6-1 cells. In some embodiments, a cell line of the invention is associated with a clinical indication, disorder or disease.

Any method known in the art can be used to measure gene expression. A preferred method is polymerase chain reaction, e.g., real-time polymerase chain reaction.

Also provided by the invention is a method of identifying the function of a test polypeptide by contacting a plurality of cells with the test polypeptide. The plurality includes a first mammalian cell, a second mammalian cell, and a third mammalian cell, wherein the first cell is a different cell type from the second cell type, the second cell type is a different cell type from the third cell type, and the third cell type is a different cell type from the first cell type. Expression of three or more genes is measured in the first cell, second cell, and third cell. An alteration in the level of expression of the gene relative to the expression of the genes in a reference cell indicates the function of the test compound. Expression is preferably measured using a polymerase chain reaction, e.g., a real-time polymerase chain reaction.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### DETAILED DESCRIPTION OF THE INVENTION

The invention provides a method for rapidly and economically identifying the function of a test agent of interest by adding the test agent to multiple cell lines, and measuring changes in gene expression of a predetermined set of genes in each cell line. By identifying those genes whose expression changes in the presence of the test agent as compared to the expression of the gene in the absence of the agent, it is possible to make inferences about the function of the polypeptide. The screen can be performed prior to, or contemporaneous with, other cell-based assays. These assays include assays measuring cell growth (bromodeoxyuridine (“BrdU”) incorporation or the calorimetric 3-(4,5)-dimethylthiazol-2-yl)-2,5-diphenyl-tetrazolium bromide (“MTT”) metabolism assay).

Cell lines and genes examined are preferably chosen so that information on changes in gene expression in a cell will provide insight into the function of the polypeptide. Examples of cells and corresponding genes suitable for use in the methods in the invention are described in Table 1. Genes for which changes in expression are associated with biological functions and relevant clinical indications are provided in Table 2. Examples of additional cells include, e.g., T Cells, monocytes, B Cells, NK Cells, normal human osteoblasts (NHOst), astrocytes, hepatocytes, and normal human lung fibroblasts. Additional genes to test for induced changes in expression are CD23, IFNγ, TNFα, and GCSF.

Screening is conveniently performed in a container in which it is possible to culture cells, add the test agent, and lyse cells for RNA isolation. The container segregates different cell types and can in addition include control cells (e.g., cells not exposed to agent). For cells whose growth is serum-dependent, the container may additionally include cells exposed to a test agent but not serum.

A preferred container is a 96-well plate. A single well of a 96-well plate generates sufficient RNA for at least 12 PCR tests, thus allowing for the probing of 11 diagnostic genes plus a negative control (where the negative control may be, for example, GAPDH minus RT) per cell line. Additionally, expression of a reference gene can be monitored in each well and serve as an internal control or standard. An example of such a reference gene is GAPDH. PCR plate layouts and cell culture techniques are commonly known within the art. Cell lysates can then be transferred to a second container, if desired, in which RNA is isolated and further manipulations (such as PCR-based analyses) performed.

Genes whose expression is to be measured are preferably chosen for each cell line to provide detection of a broad spectrum of desired biological activities, e.g., a cytokine-like activity in multiple cell types. A test compound that regulates the expression of at least one gene in at least one cell type by a factor considered to represent a significant change in the level of expression is chosen for further analysis. In one embodiment, the factor of significant change is at least ±4-fold.

The invention will be further illustrated in the following non-limiting examples.

### EXAMPLES

### Example 1

### Procedure for Assessing Polypeptide-Mediated Changes in Gene Expression in a Plurality of Cell Types

On Day 1, adherent cells are plated in a 96-well flat bottom dish in 100 μl growth medium (2×10⁴ to 3×10⁴ cells/well). On Day 2, adherent cells are washed with starvation medium and 100 μl starvation medium is added. Starvation medium contains 0.1% FBS for factor-independent cell lines (e.g., MG-63, U87-MG, HepG2, CCD-1070SK), or 2% FBS minus growth factors for factor-dependent cell lines (e.g., HUVEC). Suspension cells are plated in a 96-well round bottom dish in 100 μl starvation medium (1×10⁵ cells/well). Starvation medium contains 0.1% FBS for factor-independent cell lines (e.g., THP-1, Jurkat), and 10% FBS minus growth factors for factor-dependent cell lines (e.g., TF-1). All cells are incubated for 24 hours.

On Day 3, test compounds are added to the cells. Typically, 10 μl/well of a 10× stock for known proteins can be added. Alternatively, 10 to 100 μl/well of undiluted conditioned media for novel proteins may be used. Cells are incubated for 6 hr at 37° C. Cytoplasmic RNA is prepared from cells by centrifuging round-bottom plates containing suspension cells and discarding the supernatant. Supernatant from the flat-bottom wells containing adherent cells is also aspirated and discarded.

RLN lysis buffer is added to all sample wells. Plates are centrifuged, and the lysates (supernatants) are transferred to Uneasy columns (96 column plate). RNA is washed and eluted in 160 μl RNase free water according to the manufacturer's instructions.

On Day 4, 5, and 8, up to three plates of RNA samples are processed for TaqMan™ expression analysis. A master mix is prepared for each well as follows:

| 10× TaqMan buffer (provided by the manufacturer) | | 2.5 μl |
|---|---|---|
| MgCl₂ | 25 mM stock | 5.5 μl |
| dNTP | 2.5 mM-5.0 mM stock | 3.0 μl |
| AmpliTaq Gold | 5 U/ml | 0.125 μl |
| Multiscribe RT | 50 U/ml | 0.125 μl |
| RNAse inhibitor | | 1.0 μl |
| Forward primer | GAPDH, 10 μM stock | 0.5 μl |
| Reverse primer | GAPDH, 10 μM stock | 0.5 μl |
| Probe * | GAPDH, 5 μM stock | 0.5 μl |
| Forward primer | gene, 45 μM stock | 0.5 μl |
| Reverse primer | gene, 45 μM stock | 0.5 μl |
| Probe * | gene, 22.5 μM stock | 0.5 μl |
| dH₂O | | 2.25 μl |
| Total | | 17.50 μl |

The GAPDH or other selected reference probe is labeled according to a standard TaqMan™ protocol, e.g., 5′ ends are labeled with JOE, 3′ ends with TAMRA; while the gene-specific probes are labeled with a compound that may be monitored independently of the reference probe, e.g., 5′ ends with FAM, 3′ ends with TAMRA.

For the TaqMan™ analysis, 17.5 μl per well of the master mix is added to 96 well PCR plates containing 7.5 μl RNA sample per well. Reaction conditions include 2 minutes at 50° C., 10 minutes at 95° C., and 40 cycles of: 1 minute at 95° C., 0.40 minutes at 58° C., 1 minute at 72° C. Amplification is monitored by measuring the release of the fluorescent JOE and FAM markers during the 72° C. extension step.

Data are analyzed by comparing expression of each gene to GAPDH. To calculate changes in gene expression, gene expression in control samples is calculated and compared to the equivalent gene expression levels in the test compound-stimulated samples.

TABLE 1
Cell lines and gene lists for expression analysis.

| Cell Line, Tissue Type | Gene List | | | |
|---|---|---|---|---|
| MG-63, Osteosarcoma | IRF-1* | IL-8* | TAP-1* | LOX* |
| | OPG | Factor B* | Collagen** | Collagenase |
| | BMP-3 | MxA* | PCNA | |
| U-87MG, Astrocytoma | IRF-1*** | IL-8* | MCP-1* | ICAM-1 |
| | c-Kit | HLA-DR | iNOS | Tenascin-c* |
| | c-Myc | VEGF | GDNF | |
| TF-1, Erythroleukemia | IRF-1*** | beta-globin | EpoR | ICAM-1**** |
| | c-Kit | Factor B | GpIIb | c-Mpl |
| | GBP-2 | STAT-1 | PCNA | |
| HepG2, Hepatoma | IRF-1 | Haptoglobin | PEPCK | IGFBP1 |
| | c-Kit | CYP4A1 | Factor X | CYP7A |
| | HMGCoA Rd | Hexokinase | ApoC3 | |
| THP-1, Monocytic | IRF-1 | Egr1 | TAP-1 | ICAM-1 |
| | CCR2 | HLA-DR | iNOS | IL-12 |
| | TGF-betal | MnSOD | IL-10 | |
| HUVEC, Endothelial | PECAM | Egr1 | VCAM | ICAM-1 |
| | Tissue Factor | COX-2 | eNOS | Endothelin-1 |
| | KDR | IL-6 | MMP-2 | |
| CCD-1070SK, Fibroblast | c-Myc | IL-8 | FGF-2 | FGF-7 |
| | c-Kit | COX-2 | Factor III | Endothelin-1 |
| | HMGCoA Rd | Hexokinase | PCNA | |
| Jurkat E6-1, T-cell | IL-2 | IL-3 | IL-4 | IL-2 R |
| | CD69 | COX-2 | NFAT | Fas Ligand |
| | Bcl-2 | LFA-1 | PCNA | |

Highlighted genes were confirmed by GeneCalling on the indicated cell lines:
*= up in IL-1α treatment
**= up in OPG treatment, down in thrombopoietin treatment
***= up in IFNγ treatment
****= up in IL-6 treatment
Remaining genes were selected based on TaqMan results and literature surveys.

TABLE 2
Functional classification of gene probes.

| Functional | | | | |
|---|---|---|---|---|
| classifications: | Clinical indications: | Gene List | | |
| Angiogenesis | Cancer | PECAM | VCAM | COX-2 |
| wound healing | Surgical and burn wound | Endothelin-1 | Tissue Factor | eNOS |
| | healing | KDR | MMP-2 | IL-8 |
| | Gastric ulceration | FGF-2 | FGF-7 | VEGF |
| Inflammation | Rheumatoid arthritis | IRF-1 | ICAM-1 | MCP-1 |
| | Crohn's disease | HLA-DR | iNOS | Factor B |
| | Multiple sclerosis | GBP-2 | Haptoglobin | TAP-1 |
| | | CCR2 | IL-12 | TGF-betal |
| | | IL-10 | MnSOD | IL-6 |
| Metabolism | Obesity | CYP4A1 | IGFBP1 | PEPCK |
| | NIDDM | CYP7A | HMGCoA Rd | ApoC3 |
| | Cholesterol disorders | MxA | Hexokinase | |
| Coagulation | Thrombocytopenia | Factor X | Factor III | |
| | Hemophilia | | | |
| T-cell | Immune deficiency | IL-2 | IL-3 | IL-4 |
| activation | Cancer immunotherapy | IL-2 R | NFAT | CD69 |
| | Autoimmunity | LFA-1 | | |
| Bone | Osteoporosis | LOX | OPG | Collagen |
| formation | Bone fracture | BMP-3 | Collagenase | |
| | Growth disorders | | | |
| Growth factor | Neurodegenerative disorders | c-Kit | c-Myc | PCNA |
| Cell cycle | Cancer | Bcl-2 | Egr1 | Fas Ligand |
| Apoptosis | Autoimmunity | GDNF | Tenascin-c | |
| Hematopoiesis | Immune deficiency | beta-globin | EpoR | GpIIb |
| Erythropoiesis | Thrombocytopenia | c-Mp1 | STAT-1 | |
| | Anemia | | | |

In Table 2, many genes are associated with multiple activities, but are only listed once. For example, IL-8 could be listed in Angiogenesis and Inflammation; LOX could be listed in Bone formation and Inflammation; and Fas Test compound could be listed in Apoptosis and T-cell activation. A total of 62 distinct genes are represented.

### EQUIVALENTS

From the foregoing detailed description of the specific embodiments of the invention, it should be apparent that particular novel compositions and methods involving analysis of novel protein function have been described. Although particular embodiments have been disclosed herein in detail, this has been done by way of example for purposes of illustration only, and is not intended to be limiting with respect to the scope of the appended claims that follow. In particular, it is contemplated by the inventors that various substitutions, alterations, and modifications may be made as a matter of routine for a person of ordinary skill in the art to the invention without departing from the spirit and scope of the invention as defined by the claims. Indeed, various modifications of the invention in addition to those described herein will become apparent to those skilled in the art from the foregoing description. Such modifications are intended to fall within the scope of the appended claims.

We claim:

1. A method of identifying the function of a test compound, the method comprising
providing a plurality of cells, the plurality comprising at least a first cell and a second cell, wherein the second cell is a different cell type from the first cell type;
contacting each of the cells in the plurality with a test compound;
measuring expression of one or more genes in said first cell; and
measuring expression of one or more genes in said second cell;
wherein an alteration in the expression of said genes relative to the expression of said one or more genes in a reference cell indicates the function of said test compound.

2. The method of claim 1, wherein expression of at least two genes is measured in said first cell.

3. The method of claim 2, wherein expression of at least five genes is measured in said first cell.

4. The method of claim 2, wherein expression of at least two genes is measured in said second cell.

5. The method of claim 1, wherein s aid method further comprises measuring the expression of one or more genes in a third cell, wherein the third cell is a different cell type from the first cell and the second cell.

6. The method of claim 5, wherein said method further comprises measuring the expression of one or more genes in a fourth cell, wherein the fourth cell is a different cell type from the first cell, the second cell type, and the third cell type.

7. The method of claim 6, wherein said method further comprises measuring the expression of three or more genes in at least one of said second cell, third cell, or fourth cell.

8. The method of claim 6, wherein said method further comprises measuring the expression of three or more genes in at least two of said second cell, third cell, or fourth cell.

9. The method of claim 6, wherein said method further comprises measuring the expression of three or more genes in at least three of said second cell, third cell, or fourth cell.

10. The method of claim 1, wherein said cells are provided in a container.

11. The method of claim 1, wherein expression of one or more of said genes is compared to expression of a reference gene.

12. The method of claim 11, wherein said test compound modulates expression of said one or more genes at least four-fold relative to said reference gene.

13. The method of claim 1, wherein said test compound is a polypeptide.

14. The method of claim 1, wherein said method comprises contacting at least some cells in said plurality with two or more test compounds.

15. The method of claim 1, wherein said plurality of cells comprises mammalian cells.

16. The method of claim 15, wherein said cells are human cells.

17. The method of claim 1, wherein said first cell is selected from the group consisting of MG-63 cells, U87-MG cells, TF-1 cells, HepG2 cells, THP-1 cells, HUEC cells, CCD-1070SK cells, and Jurkat E6-1 cells.

18. The method of claim 1, wherein expression of one or more sequences is measured using real-time polymerase chain reaction.

19. A method of identifying the function of a polypeptide test compound, the method comprising
providing a plurality of cells, the plurality comprising at least a first mammalian cell, a second mammalian cell, and a third mammalian cell, wherein the first cell is a different cell type from the second cell type, the second cell type is a different cell type from the third cell type, and the third cell type is a different cell type from the first cell type;
contacting each of the cells in the plurality with said polypeptide;
measuring expression of three more genes in said first cell;
measuring expression of three or more genes in said second cell; and
measuring expression of three or more genes in said third cell;
wherein an alteration in the expression of said genes relative to the expression of said genes in a reference cell indicates the function of said test compound.

20. The method of claim 19, wherein expression of said genes is measured using real-time polymerase chain reaction.
